(19) United States
(12) Patent Application Publication
(10) Pub. No.: US 2012/0288538 A1
(43) Pub. Date: Nov. 15, 2012

(54) VACCINE AGAINST NEOPLASTIC OR CANCEROUS LESIONS CAUSED BY HUMAN PAPILLOMA VIRUS (HPV), PROCEDURES, USES AND METHODS

(75) Inventors: Gonzalo De Prat Gay, Capital Federal (AR); Maria Laura Cerutti, Capital Federal (AR); Alonso Leonardo Gabriel, Capital Federal (AR)

(73) Assignees: CONSEJO NACIONAL DE INVESTIGACIONES CIENTIFICAS Y TECNICAS, Buenos Aires (AR); INIS BIOTECH LLC, Milford ,Kent County, DE (US); FUNDACION INSTITUO LELOIR, Buenos Aires (AR)

(21) Appl. No.: 13/513,696

(22) PCT Filed: Dec. 2, 2010
(86) PCT No.: PCT/US2010/058657

§ 371 Date: Jul. 30, 2012

(30) Foreign Application Priority Data
Dec. 4, 2009 (AR) 20090104714

(51) Int. Cl. A61K 9/14; C07K 1/107; A61P 35/00; A61P 31/20; A61P 17/12; A61K 39/12; A61P 37/04

(52) U.S. Cl. 424/400; 424/204.1; 530/345

(57) ABSTRACT

Vaccine against neoplastic or cancerous lesions caused by human papillomavirus (HPV), which comprises E7 peptide spherical particles and, as an option, an adjuvant, where spherical particles may be oligomeric. The oligomeric spherical particles may have a diameter in the vicinity of 50 nm and a molecular weight in the vicinity of 700 kDa. The vaccine may be helpful to prevent or treat human papillomavirus (HPV)-related lesions or do both things at the same time.

The invention is related to a vaccine against neoplastic or cancerous lesions caused by the human papillomavirus (HPV). More specifically, it relates to a vaccine comprising E7-peptide spherical particles of said virus and optionally an adjuvant, where the spherical particles are oligomeric. The oligomeric spherical particles may have a diameter in the vicinity of 50 nm and a molecular weight in the vicinity of 700 kDa. Vaccines according to the invention may be useful to prevent or treat papillomavirus (HPV)-related lesions.

### BACKGROUND OF THE INVENTION

The HPV is an etiological agent for cervical cancer, the second cause of mortality among cancer-affected women in the world. Estimations say that every year there are half a million cases of cervical cancer approximately, 80% of which occur in developing countries because of a lack of routine examinations of the populations (for example, Papanicolau smear, or Pap).

Of the more than 100 HPV genotypes found to date, 40% infect mucosal areas. A persistent infection by high-risk HPV genotypes is the necessary factor for the development of cervical cancer (>95% of the cases), but this cancer is also associated to other anogenital tumors, such as vaginal (65-90%), vulval (40%), penis (40%) and anal canal (90%) ones and, in a lesser proportion, to oral cavity tumors (<30% of cases) (Parkin, D. M. and Bray, F. (2006) Chapter 2: The burden of HPV-related cancers. Vaccine, 24 Suppl 3, S11-25). More than half of cervical cancers are caused by the HPV 16 genotype which, together with the high-risk types HPV 18, 31 and 45, represent nearly 80% of all the cases (Clifford, G. et al., (2006) Chapter 3: HPV type distribution in women with and without cervical neoplastic diseases. Vaccine, 24 Suppl 3, S26-34). Low-risk genotypes such as HPV 6 and 11 cause benign genital warts, this being, perhaps, the most common of all sexually transmitted diseases.

Extant prophylactic vaccines have proven to be highly efficient to generate a humoral immunity protecting against infections from most high-risk prevalent genotypes, such as HPV 16 and 18. However, in spite of a ˜100% efficiency to prevent HPV infections, these vaccines have no therapeutic effect on pre-existing neoplastic processes and, consequently, they do not have an immediate impact on cervical cancer incidence (Kols, A., et aL, (2006). PATH, Seattle, Wash. USA and Leggatt, G. R. and Frazer, I. H. (2007) Curr Opin Immunol, 19, 232-238).

Cervical cancer results from a spectrum of precursor lesions very well defined histologically: cervical intraepithelial neoplasias (CIN). This type of lesions is routinely detected in the course of cytological exploration programs and usually removed surgically or destroyed via laser therapy or cryotherapy. Since many affected women are still in the reproductive age and these procedures entail a certain degree of associated morbidity and may even lead to infertility, alternative treatments are peremptory.

Follow-up studies have shown that a persistent infection is a prerequisite for the development of a high degree CIN and that oftentimes the infection precedes clinical symptoms for several years. Therefore it is assumed that there exists a valuable and extended time window for cervical cancer treatment, even in postexposure situations (Michel, N., et al., (2002) Intervirology, 45, 290-299).

Therapeutic vaccines may be used to treat established HPV infections and consequently they might have an immediate effect on the prevalence of HPV-associated malignancies. Therapeutic vaccination strategies attempt to eliminate preexisting lesions and also malignant tumors, through the generation of cell-mediated immunity against infected cells. Thus, in order to eliminate existing lesions, a therapeutic vaccine should aim at HPV antigens constitutively expressed in infected transformed cells. HPV early proteins, E6 and E7, for a number of reasons constitute ideal targets for this purpose: firstly, they are constitutively expressed in HPV tumors. Secondly, since E6 and E7 are crucial for the induction and maintenance of cell transformation into HPV-infected cells, it is unlikely that tumor cells be able to escape an immune attack through antigen loss. Thirdly, since both are foreign proteins, they permit to avoid some of the common problems associated to cancer vaccines, such as immune tolerance (Hung, C. F., et al., (2008) Expert Opin. Biol. Ther., 8, 421-439). Of these two oncogenic viral proteins, the most relevant one is the E7 peptide.

In recent years several reports have shown the therapeutic efficiency of E7 from HPV-16-based vaccines, in preclinical and clinical studies. These HPV vaccine candidates include recombinant live vectors carrying the E7 gen, purified E7 protein, E7-HLA peptide epitopes and E7 expression plasmids. In many of these cases, the strategy also includes the fusioning of the E7 protein, peptide or gene with another known molecule that increases inflammation or immunity (Hung, C. F., et al., (2008) Expert Opin. Biol. Ther., 8, 421-439). Notwithstanding the extraordinary improvements that took place in the last decade on DNA-based vaccine technology and the promising results achieved with experimental models, both the scarce immunogenicity in superior organisms and ethical issues still are their main drawbacks. In this respect, protein-based vaccines still constitute the first choice, by offering safety, a relatively low cost and high immunogenicity. Besides, the use of last-generation adjuvants may modify even more the candidate protein immunogenic properties according to the need to favor a humoral protection of a host or a T cell-mediated therapy. Through the use of these adjuvants, tedious technical steps and excesses of undesirable physiological reactions to fusioned immunostimulating genes can be avoided.

E7 is a small acidic peptide bound to zinc via its c-terminal domain. Generally, the therapeutic efficiency of HPV-vaccine candidates is tested on a cervical cancer-Tc1 murine tumor, which resembles the HPV16-tumor phenotype. The Tc1 cell line derives from C57BL/6-mice primary epithelial cells immortalized with E6 and E7 genes from HPV16 and transformed with the c-Ha-ras oncogene (Lin, K. Y.,et al., (1996) Cancer Re.s, 56, 21-26.). This cotransformation created a tumorigenic cell line expressing E6 and E7 oncoproteins, mimicking the natural sequence to cervical cancer progression, where these oncoproteins immortalize the cells and subsequent mutations into cell proto-oncogenes transform them into tumor cells with metastatic potentiality. The resulting Tc1 cell line has a high mitotic rate and a rapid growth. Experiments on tumor growth showed that a subcutaneous inoculation of 5×10⁴ cells suffices to produce 100% tumors in mice within the 20 days following the injection (Lin, K. Y., et al., (1996) Cancer Res., 56, 21-26).

### BRIEF DESCRIPTION OF THE INVENTION

The invention is related to vaccines against cancerous lesions caused by the HPV; these vaccines comprise E7-peptide spherical particles of the papilloma virus and, as an option, an adjuvant; the spherical particles may be oligomeric. The oligomeric spherical particles may have a diameter in the vicinity of 50 nm and a molecular weight in the vicinity of 700 kDa. Vaccines according to the invention may prevent or treat HPV-related lesions.

It is also shown a procedure to stabilize said spherical particles, which comprises the oxidation of said particles. In a preferred embodiment, the oxidation is carried out by putting into contact the spherical particles and copper sulphate; to this follows an incubation and a subsequent removal of the remaining copper.

In addition, it is shown the use of said spherical particles to prepare a medication for treating lesions caused by HPV.

It is shown a method for treating HPV-lesion carriers, method comprising the administration of a sufficient amount of a vaccine comprising spherical particles of HPV E7 peptide to an individual

It is shown a method for immunization against HPV, method comprising the administration of a sufficient amount of a vaccine comprising spherical particles of papillomavirus E7 peptide to an individual

### DESCRIPTION OF FIGURES

FIG. 1 shows the results of a preventive vaccination with E7-MPL and E7SO-MPL according to example 1; FIG. 1A shows the tumoral volume of each one of the mice vaccinated with MPL, E7-MPL, E7SO-MPL and E7SO on the days shown in the follow-up. *This mouse showed a general condition of bad health and an anomalous position of the tumor. FIG. 1B shows with a curve the free tumor-development period for each treatment (E7-MPL, circles; E7SO-MPL, squares; MPL, triangles and E7SO, crosses);

FIG. 2 shows the results of the treatment of Tc1 tumor-carrying mice, according to example 2, with the compositions comprising E7-MPL particles (circles) or E7SO-MPL spherical particles (squares) and MPL as the control (triangles), where tumor volume growth can be seen as a function of post treatment days;

FIG. 3 shows the results of the treatment of Tc1 tumor-carrying mice, according to example 2, with the compositions comprising E7-MPL dimeric particles (circles) or E7SO-MPL spherical particles (squares) and MPL as the control (triangles), as a function of the survival of the tumor-carrying animal.

FIG. 4 shows the therapeutic capability of the vaccine comprising the E7 (E7SO) oligomeric particles combined with the adjuvant ODN2006. This capability was assessed by measuring the tumor size.

### DETAILED DESCRIPTION OF THE INVENTION

In this description, the term vaccine is to be construed as composition having a protecting or a therapeutic activity, or both at the same time, against HPV lesions.

In this description, it is to be construed that the acronym E7 corresponds to a vaccine comprising E7 dimeric particles. E7-MPL corresponds to a vaccine comprising E7 dimeric particles and MPL adjuvant. E7SO corresponds to a vaccine comprising E7 oligomeric spherical particles and MPL adjuvant. Surprisingly, the oligomeric spherical particles showed high antitumor activity.

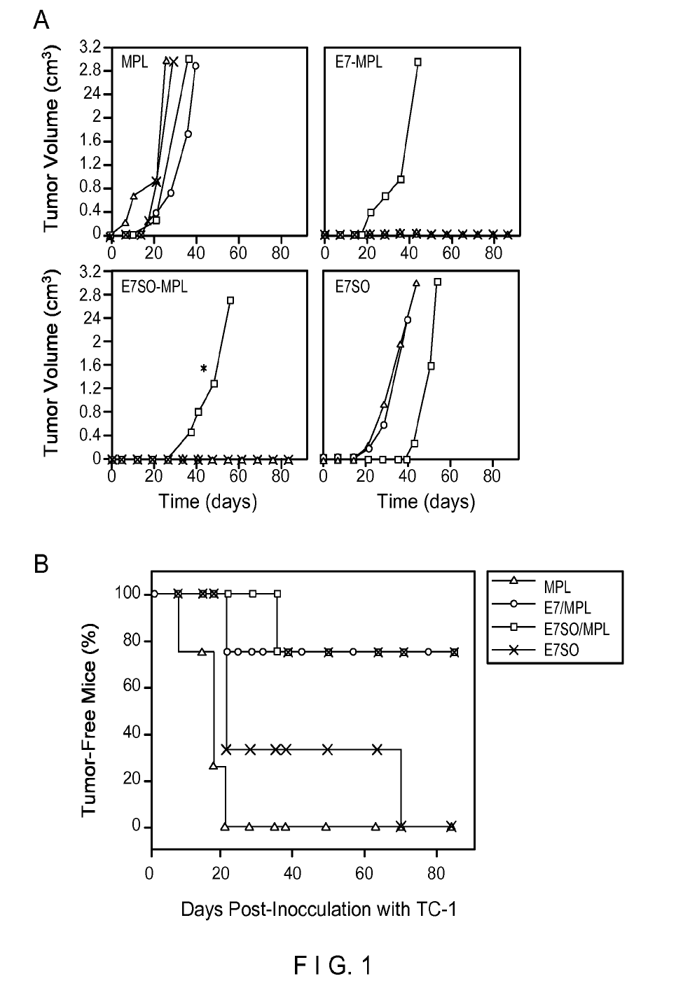

The preventive antitumor effect of the vaccine according to the invention, that comprises E7 oligomeric spherical particles (E7SO) and optionally an adjuvant was assessed. Particle capability of protection against the Tc1 tumor cell line expressing HPV16 E7 was assessed. On the basis of that, seven days after the last administration of the vaccine dose (day 28), all of the immunized mice were challenged with Tc1-cell lethal doses. FIG. 1 shows the tumor growth curves for different groups of vaccinated mice.

According to these data, 100% of the mice immunized with MPL adjuvant develop large aggressive tumors within 7-15 days after challenge; all of the mice were sacrificed on day 35 (FIG. 1A and B). In contrast, an administration of the vaccine according to the invention, that comprises the oligomeric spherical particles with an adjuvant (E7SO-MPL, for example MPL) prevented tumor development in 80% of mice, which remained tumor-free after being followed up for 90 days. Besides, in the two mice that developed tumors, the apparition of said tumor was delayed 10-20 days, with respect to control mice vaccinated with MPL only.

Interestingly, although all E7SO-immunized mice developed tumors, they did it with a slight delay with respect to the development shown by the control group with MPL, what suggests that E7SO might have a certain antitumor effect, even in the absence of the adjuvant.

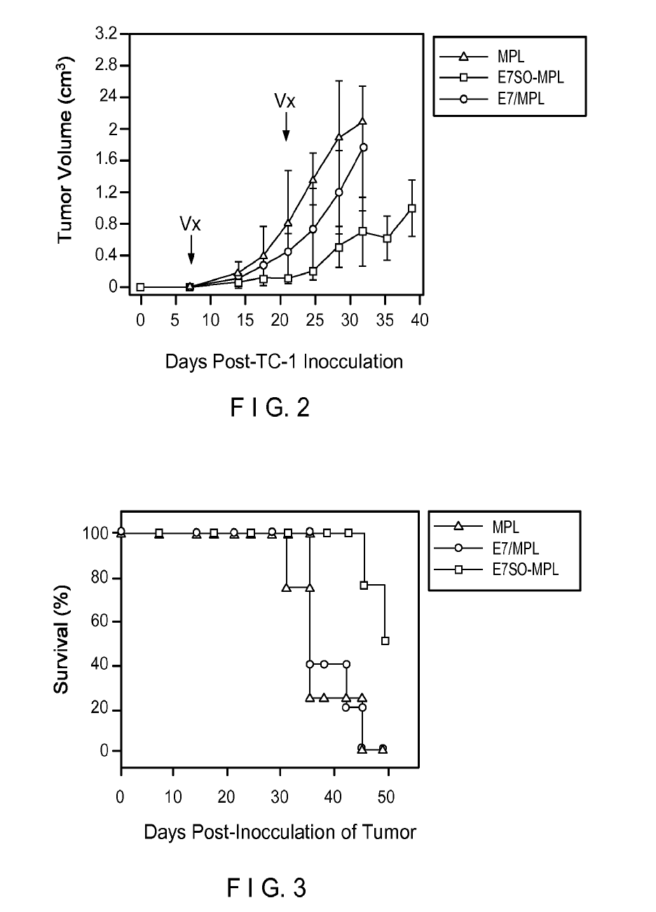

Once shown the capability of the vaccine according to the invention, that comprises the spherical particles plus an adjuvant (E7SO-MPL), to protect challenged animals having a Tc1 tumor, the activity of said vaccine for the treatment of tumor-carrying mice expressing the HPV16 E7 oncoprotein was assessed. In this case, female mice were inoculated subcutaneously with 5×10⁴ Tc1 cells and, once the tumor was palpable (day 7), the mice were treated with the vaccines comprising E7-MPL or E7SO-MPL. A second dose was given two weeks later (day 21). As shown in FIG. 2, the inoculation of the vaccine according to the invention significantly delays tumor growth. On day 32, when most of the MPL-vaccinated mice (control) had to be sacrificed because of the extension of the tumor, the average volume of the tumor in E7SO-MPL-vaccinated mice was four times smaller (˜2,3 cm³ vs ˜0,6 cm³, respectively). Moreover, mice vaccinated with E7-MPL, on day 32 also showed a tumor size increase similar to that of the control and nearly four times larger than the size of tumors in vaccine E7SO-MPL-treated animals.

The difference between treatments is also displayed in the survival rate of the different groups treated, which was 100% for the group treated with E7SO-MPL, and 0% for the MPL group (control) on day 35 (FIG. 3). In the experiments on protection against tumors it is furthermore shown that the treatment with the vaccine comprising E7 dimeric particles has a lesser therapeutic effect (FIGS. 2 and 3), this indicating that in tumor-carrying female mice, the E7 oligomerization state is critical for the induction of an effective antitumor response mediated by cytotoxic T cells

The inoculation of female mice with low doses of the vaccine according to the invention, for example with the E7SO-MPL vaccine, induced a complete humoral and cellular immune response, inducing titers of anti E7 serum specific IgG and protective immunity against E7-expressing Tc1 tumor cells. Moreover, the vaccination of tumor-carrying female mice with the E7SO-MPL vaccine decelerated the exorbitant tumor growth and extended the survival period, thus showing the vaccine potential as immunotherapeutic agent.

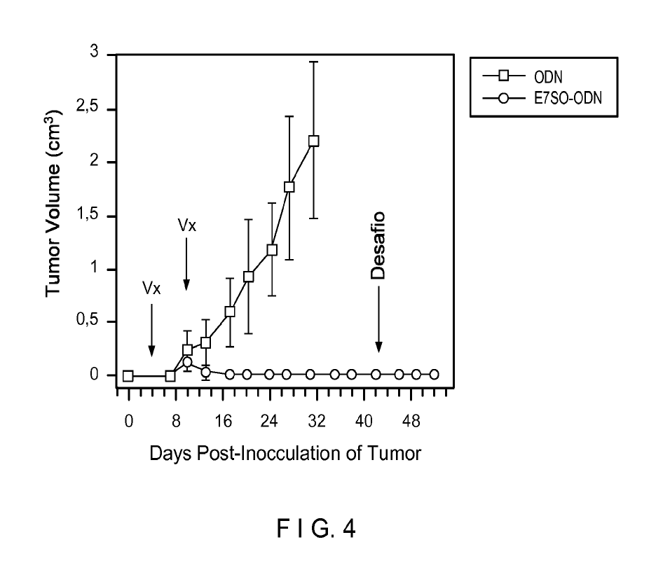

As can be seen in FIG. 4, E7SO oligomeric spherical particles have a high therapeutic effect, as compared to dimeric E7, for example. The therapeutic capability of the vaccine comprising E7 oligomeric particles combined with an oligodeoxynucleotide of proven efficiency as adjuvant in human cells, ODN2006 was assessed (Hartmann, G. et al., (2000) J. Immunol., 164,1617-1624).

The vaccine according to the invention may be combined with an adjuvant such as the 3-deacylated monophosphoryl lipid A (MPL) adjuvant, which is a non-toxic derivative of the lipopolysaccharides (LPS) in Gram-negative bacteria wall, or with the ODN adjuvant. The experts in the vaccine-production art know that any adjuvant may be used, all of the adjuvants being within the scope of this invention. In a preferred embodiment, the adjuvant is MPL or ODN.

The antigenic properties of the vaccines according to this invention were assessed by means of an ELISA dosage of the titer of vaccinated-mice serum antibodies. The immunization with the S7SO-MPL vaccine according to the invention results in specific titers of IgG antibodies against the E7 high-risk peptide (˜1/6000).

In an embodiment of the invention it is shown a purification procedure permitting the obtainment of great amounts of chemically pure E7 protein that was recombinantly expressed in Escherichia coli and it was demonstrated that the main species was a dimer with a molecular weight of 22 kDa. In another embodiment of the invention it is shown a procedure for the obtainment of a protein that can be assembled into homogeneous spherical particles having an average molecular mass of 790 kDa and a diameter of 50 nm. The assembly is a very slow process, where the protein undergoes substantial conformational transitions with a concomitant consolidation of its tertiary structure. The resulting particles (E7SO) are highly stable, cooperatively pleated and they resemble the β sheet structures found in soluble or insoluble amyloids.

The invention is better illustrated by the following examples, which should not be construed as a limitation for the extent and scope of the invention: on the contrary, it must be clearly understood that it may be resorted to whatever embodiments, modifications and equivalents of the invention that after the reading of this description might be suggested to the experts in this art, without departing from the spirit of this invention or the scope of the appended claims or without doing any of these two things

### Examples

### Example 1

### Experiments on on Vivo Prevention

Groups of mice (n=5) were randomly distributed in the diverse groups and vaccinated twice intraperitoneally at 21-day intervals, with 50 g of E7 dimers or highly purified E7 soluble oligomers and 25 g of MPL adjuvant (groups E7-MPL and E7SO-MPL, respectively); 50 g of E7 soluble oligomers without adjuvant (group E7SO) or 25 g of MPL alone (group MPL). Seven days after the last reinforcement (day 28), the mice were subjected to an exploratory bleeding and then subcutaneously inoculated (s.c.) on their left sides with 5×10⁴ Tc1cells. In all of the experiments, the viability of tumor cells implanted in the mice was >90%. Tumor growth was measured twice per week using an electronic caliper and tumor volume was calculated as (length×width2)/2. The animals were sacrificed when the size of the tumors was 3 cm³, approximately.

### Example 2

### Experiments on Treatment

For therapeutic experiments, the mice were first challenged s.c. on their left sides with 5×10⁴ Tc1 tumor cells (day 0). When all of the animals had palpable tumors (day 7), they were arbitrarily assigned to groups (5 per group) and vaccinated i.p. with E7-MPL, E7SO-MPL or MPL alone. The vaccine doses were the same as those used in example 1. A second reinforcement was given on the day 21. The animals were sacrificed when their tumors reached a size of 2,5 cm³.

### Example 3

Preparation of E7 dimers and E7SO Oligomers Used in the Vaccine

The E7 from HPV16 was purified as described (Alonso, L.G., et al., (2002). Biochemistry, 41, 10510-10518). E7SO was prepared from high-purity E7 dimer particles that were incubated as previously described (Alonso, L.G., et al., (2004). Biochemistry, 43, 3310-3317). Afterwards, to a 40 uM solution of E7SO in sodium phosphate 10 mM pH 7.0, it is added copper sulphate up to a final concentration of 20 uM and it is incubated at 28° C. for 24 hours. After the oxidation, the sample is dialized against buffer 10 mM pH 7.0 of sodium phosphate, to remove the excess of Cu. The oligomer oxidation state is assessed in a SDS-PAGE 15% without a reducer.

### Example 4

### Production of the Vaccine

The aqueous solution of MPL adjuvant was prepared as described (Baldridge, J. R and Crane, R. T., (1999). Methods, 19, 103-107) and it was subsequently kept at 4° C. The dimeric and oligomeric particles of E7 HPV16, diluted in PBS buffer up to the desired final concentration (0.25 ug/ul), were mixed with the MPL adjuvant in the proportions described in the example 1 at the time of administration. The final volume given to each animal was 200 ul.

### Example 5

Production of the Vaccine with Adjuvant ODN

The aqueous solution of ODN2006 adjuvant was prepared as described (Hartmann, G. et al., (2000) J. Immunol., 164,1617-1624), and it was subsequently kept at −20° C. The E7SOs particles (60 ug doses) were diluted in PBS buffer and mixed with the ODN2006 adjuvant (30 ug doses) at the time of administration. The final volume given to each animal was 200 ul.

### Example 6

Experiments on Treatment with ODN

Therapeutic experiments were carried out on female mice first inoculated s.c in their left sides with 5×10⁴ Tc1 tumor cells (day 0). After four days (day 4), the mice were arbitrarily assigned into groups and s.c. vaccinated in the base of their tails with 60 g of highly purified E7 soluble oligomers and 30 g of ODN2006 adjuvant (group E7SO-ODN, 8 animals per group) or with adjuvant alone (group ODN, 4 animals per group). A second reinforcement was given on day 11. The animals were sacrificed when the tumor size was 2.5 cm³.

1-16. (canceled)

17. A vaccine against neoplastic or cancerous lesions caused by the human papillomavirus (HPV), comprising oligomeric spherical particles of the human papillomavirus E7 peptide.

18. The vaccine of claim 17, further comprising an adjuvant.

19. The vaccine of claim 17, wherein the spherical particles have a diameter equal to, or larger than, 15 nm.

20. The vaccine of claim 17, wherein the spherical particles have a molecular weight equal to, or higher than, 50 kDa.

21. The vaccine of claim 18, wherein the adjuvant is the lipid A monophosphoril 3-deacylated (MPL).

22. The vaccine of claim 18, wherein the adjuvant is ODN2006.

23. The vaccine of claim 17, wherein the oligomeric spherical particles comprise between 2 and 100 E7 peptide monomers.

24. The vaccine of claim 17, wherein it is used for the prevention of HPV-associated lesions.

25. The vaccine of claim 17, wherein it is used for the treatment of HPV-associated lesions.

26. A procedure to stabilize the spherical particles of claim 17, comprising a controlled oxidation of said particles.

27. The procedure of claim 26, comprising
a) the contacting of the spherical particles with copper sulphate and subsequent
b) incubation; and
c) the removal of remaining copper.

28. A method for the treatment of HPV-associated lesion carriers, comprising the administration of a sufficient amount of a vaccine comprising spherical particles of papillomavirus E7 peptide to an individual.

29. The method according to claim 28, wherein the HPV-associated lesion is selected of group consisting of skin warts, genital warts, flat warts, epidermodysplasia verruciformis, non-melanoma skin cancer, condylomata acuminate, venereal warts, cervical, vulvar, penile and anal intraepithelial neoplasias, recurrent respiratory papillomatosis, tongue cancer, tonsils cancer and throat cancer.

30. A method for immunizing against HPV-associated lesions, comprising the administration of a sufficient amount of a vaccine comprising spherical particles of papillomavirus E7 peptide to an individual.

31. The method according to claim 30, wherein the HPV-associated lesion is selected of group consisting of skin warts, genital warts, flat warts, epidermodysplasia verruciformis, non-melanoma skin cancer, condylomata acuminate, venereal warts, cervical, vulvar, penile and anal intraepithelial neoplasias, recurrent respiratory papillomatosis, tongue cancer, tonsils cancer and throat cancer.
